(19) United States
(12) Patent Application Publication
(10) Pub. No.: US 2005/0106734 A1
(43) Pub. Date: May 19, 2005

(54) Fungal micro-organism having an increased ability to carry out biotechnological process(es)

(76) Inventors: Peter Richard, Helsinki (FI); John Londesborough, Helsinki (FI); Merja Penttila, Helsinki (FI); Ritva Verho, Espoo (FI)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/494,030

(22) PCT Filed: Oct. 29, 2002
(86) PCT No.: PCT/FI02/00841

(30) Foreign Application Priority Data
Oct. 29, 2001 (FI) 20012091

(51) Int. Cl. C12N 1/18; C12N 15/74

(52) U.S. Cl. 435/483; 435/254.2

(57) ABSTRACT

The present invention relates to fungal microorganism having an increased ability to carry out biotechnological process(es). In particular, the invention relates to improving the regeneration of redox cofactors in biotechnological processes where useful products are produced from biomass containing pentoses. According to the invention, the microorganism is transformed with a DNA sequence encoding an NADP linked glyceraldehyde 3-phosphate dehydrogenase. The invention can be used to provide useful products for mankind from biological materials, including e.g. agricultural and forestry products, municipal waste. Examples of such useful products are ethanol, lactic acid, polyhydroxyalkanoates, amino acids, fats, vitamins, nucleotides and a wide variety of enzymes and pharmaceuticals.

### FIELD OF THE INVENTION

The present invention relates to fungal microorganism having an increased ability to carry out biotechnological process(es). In particular, the invention relates to improving the regeneration of redox cofactors in biotechnological processes where useful products are produced from biomass containing pentoses.

### BACKGROUND OF THE INVENTION

This application is concerned with the efficiency of biotechnological processes, meaning industrial processes that use the metabolic reactions of microorganisms, especially yeasts and other fungi, to provide useful products for mankind from biological materials, including agricultural and forestry products, municipal waste and other biomass sources. Examples of such useful products are ethanol, lactic acid, polyhydroxyalkanoates, amino acids, fats, vitamins, nucleotides and a wide variety of enzymes and pharmaceuticals.

Some metabolic reactions are coupled to the redox cofactor couple nicotinamide dinucleotide phosphate/reduced nicotinamide dinucleotide phosphate (NADP/NADPH) others to the redox cofactor couple nicotinamide dinucleotide/reduced nicotinamide dinucleotide (NAD/NADH). In general, the cofactors NAD/NADH are mainly related to catabolic reactions, the cofactors NADP/NADPH mainly to anabolic reactions. Usually the productive pathways of biotechnology processes give excess NADP. Pentose fermentation is one example of that. In pentose fermentation, through the L-arabinose and the D-xylose pathways, some catabolic reactions are coupled to the NADP/NADPH cofactors (see FIG. 1).

The fermentation of D-xylose to ethanol (or lactic acid) is redox neutral but different redox cofactors are used, which creates a redox cofactor imbalance. The xylose reductase utilises NADPH and produces NADP. The other redox steps are xylitol dehydrogenase, glyceraldehyde 3-phosphate dehydrogenase and alcohol dehydrogenase, each of them utilising the NAD/NADH redox cofactor couple. As a consequence of this redox cofactor imbalance, NADPH must be regenerated by other reactions e.g. the oxidative part of the pentose phosphate pathway which is coupled to CO₂ production. CO₂ is an unwanted product and the conversion of D-xylose to ethanol (or lactic acid) is not anymore redox neutral (FIG. 2). As a consequence also other unwanted products such as xylitol are produced.

Therefore, it would be beneficial to be able to regenerate the NADP(H) cofactors, in particular in a fungal pentose (D-xylose and L-arabinitol) fermentation. An efficient way to regenerate the NADP(H) cofactors would be of biotechnological benefit since it would make the process less dependent on strict oxygen control, reduce the need of oxygen or facilitate anaerobic pentose (D-xylose and L-arabinose) fermentation. Anaerobic pentose fermentation is very slow and unwanted side products are produced; semi-anaerobic conditions are required for optimal fermentation conditions (Jeffries and Jin, 2000). This would in practise require a controlled aeration, i.e. a technically complicated process. The products of pentose fermentation are in general cheap bulk products (such as ethanol). This would require a cheap production process, such as anaerobic fermentation. Anaerobic fermentation is technically easy and can be done in very large scale. However with the current technology anaerobic D-xylose fermentation leads mainly to unwanted side products such as xylitol and CO₂ (Toivari et al. 2001). The production of xylitol and CO₂ from D-xylose is redox neutral. The stochiometry for a redox neutral conversion is 10 moles of xylitol and 5 moles of CO₂ are produced from 11 moles of D-xylose. It is apparently the preferred reaction over the production of ethanol and CO₂ in cases where the NADP, which is produced in the D-xylose reduction, is reduced to NADPH in reactions producing CO₂. If the NADP could be reduced in a way which is not directly linked to CO₂ production it could lead to a redirection of the products from xylitol and CO₂ to ethanol and CO₂, and hence a more economic (cheaper) process with environmental benefits.

In patent application WO 99/46363 (Aristidou et al.) production microorganisms used in biotechnology were disclosed with improved properties that produce useful products, such as ethanol and amino acids, more efficiently. A microorganism was provided which is transformed with at least one recombinant DNA molecule encoding an oxidoreductase, so that a pair of oxidoreductases with at least one common substrate but different coenzyme specificities for NAD/NADH and NADP/NADPH are expressed in such a way that both members of the pair are simultaneously expressed in the same sub-cellular compartment, preferably the cytosol. This results in introduction of a transhydrogenase activity through cyclic oxidation and reduction reactions with different cofactors.

The cyclic oxidation and reduction reactions allow the following reactions to occur, which tend to equilibrate the NAD/NADH and NADP/NADPH coenzyme couples:
NADP+SH₂⇄S+NADPH (1)
S+NADH⇄SH₂+NAD (2)

Simultaneous operation of reactions (1) and (2) might be expected to proceed until the NAD/NADH and NADP/NADPH ratios are almost identical, because the redox potentials of the two couples are very similar.

In patent publication U.S. Pat. No. 5,830,716, a method for production of a target substance using a microorganism is disclosed. In this method, the microorganism has been modified so that its ability to produce reduced nicotinamide adenine dinucleotide phosphate (NADPH) from reduced nicotinamide adenine dinucleotide (NADH) is increased, whereby production of the amount of the target substance, such as L-amino acid, is increased in the culture medium. The ability of the microorganism to produce NADPH from NADH is increased by increasing the nicotinamide nucleotide transhydrogenase activity of the microorganism.

More efficient systems to regenerate NADPH are still needed to improve the many biotechnological processes where the main metabolic pathway from substrate to product produces net NADP. Such processes include the fermentation of pentoses to ethanol, lactate and other products and the production of polyalkanoates, some amino acids and lipids from carbohydrates.

### SUMMARY OF THE INVENTION

The object of the present invention is to provide a fungal microorganism having an increased ability to carry out biotechnological process(es). This is achieved according to the invention by transforming a fungus with a gene coding for an NADP-linked glyceraldehyde 3-phosphate dehydrogenase (NADP-GAPDH;EC 1.2.1.13). With the gene product NADPH is regenerated in a catabolic reaction which is beneficial in pentose fermentation or other processes.

Preferably, the NADP-GAPDH is of fungal origin and the DNA sequence encoding it comprises SEQ ID No. 1 or a functional variant thereof The invention provides industrial microorganisms transformed with a DNA sequence encoding an NADP-linked GAPDH so that the transformed microorganisms have a novel means of regenerating the reduced, NADPH, form of the NADP/NADPH coenzyme couple. In yeasts and other fungi and most other microorganisms GAPDH is a step on the main metabolic route by which sugars are converted to pyruvate and onward to cell material and fermentation end products. The transformed microorganism of the invention has two GAPDH enzymes, one that works with NAD and another that works with NADP. The transformed organisms automatically adjust the relative fluxes through these two enzymes in order to regenerate NADPH and NADH as demanded by other metabolic steps.

Using genetic engineering techniques it is also possible to modulate the relative expression levels of the genes encoding NADP and NAD-linked GAPDH enzymes so that, for instance, in the conditions used for product formation the level of the NAD-linked enzyme is decreased or practically omitted, thus leading to increased use of NADP in the GAPDH reaction. A transformed microorganism of the invention leads to more efficient biotechnological processes where the desired reactions (e.g., conversion of pentoses to ethanol or lactate; conversion of sugars to lipids or amino acids or polyhydroxyalkanoates) are net consumers of NADPH, because in the transformed microorganism NADPH can be regenerated by the introduced NADP-linked GAPDH, which is a step in the main metabolic pathway used by the desired process itself thus decreasing or eliminating the need to regenerate NADPH by side reactions (for example the oxidative branch of the pentose phosphate pathway) that waste carbon substrate, or have limited capacity or both. The expression ‘more efficient biotechnological processes’ encompasses industrial processes that have a higher yield of desired product on substrate, a greater volumetric productivity (measured as mass of product per unit time per unit reactor volume), a greater specific rate (measured as mass of product per unit time per unit mass of production microorganism), produce smaller amounts of undesired side products, can be operated more cheaply, for example in simpler fermentors or with less aeration, or have two or more of these benefits.

The invention provides a DNA sequence that encodes an NADP-linked GAPDH from Kluyveromyces lactis that can be used to practise the invention. The invention also provides methods to find other DNA sequences that encode proteins with NADP-linked GAPDH activity and can be used to practise the invention. Further, certain characteristics of the amino acid sequences of NADP-linked GAPDH are disclosed that enable a person skilled in the art to recognise DNA sequences that encode proteins with NADP-linked GAPDH activity that can be used to practise the invention, or to engineer such DNA sequences conveniently from DNA sequences that encode proteins with NAD-linked GAPDH activity.

The invention provides a suitable constitutive promoter that can be used to drive the expression of an NADP-linked GAPDH for the purposes of the invention. However, other promoters can be used and it is envisioned that for some hosts and bioprocesses it may be advantageous to express the NADP-linked GAPDH from an inducible or repressible promoter.

The present invention is now explained in detail by referring to the attached figures and examples. These examples are only used to show some of the embodiments and are not intended to limit the scope of the invention.

### BRIEF DESCRIPTION OF THE DRAWINGS

FIG. 1. The fungal pathways for L-arabinose and D-xylose. L-arabinose is converted to D-xylulose 5 phosphate in a pathway which includes 2 reduction and 2 oxidation steps. The reduction steps are coupled to the oxidation of NADPH, the oxidation steps to a reduction of NAD. D-xylose is catabolised in a similar way including 1 reduction and 1 oxidation. Also here the reduction is coupled to an oxidation of NADPH and the oxidation to a reduction of NAD.

FIG. 2. The redox cofactors in the D-xylose fermentation. The fermentation of 3 moles of D-xylose to 5 moles of ethanol and 5 moles CO₂ is redox neutral. However different redox cofactors are used, i.e. NADP and NADH are not sufficiently regenerated, creating an imbalance of redox cofactors. NADP can be regenerated, e.g. by the oxidative part of the pentose phosphate pathway. This would lead to an extra CO₂ production so that the overall process is not anymore redox neutral.

FIG. 3. The redox cofactors in the D-xylose fermentation with an NADP-GAPDH. The conversion of 3 moles of D-xylose to 3 moles of D-xylulose results in the production of 3 moles of NADP and 3 moles of NADH. From 3 moles D-xylulose 5 moles of glyceraldehyde 3-phosphate (GAP) can be produced. 3 moles of GAP can recycle the 3 moles of NADP back to NADPH. The other two moles of GAP are used to reduce 2 moles NAD to NADH. In total 5 moles of NADH were produced which can be regenerated by the alcohol dehydrogenase to produce ethanol. The production of 5 moles of ethanol and 5 moles CO₂ is now cofactor neutral.

FIG. 4. Ethanol and xylitol production during anaerobic D-xylose fermentation in the strain overexpressing the NADP-GAPDH (full symbols) and the corresponding control without NADP-GAPDH activity (open symbols). The concentrations are given in mM per g of dry weight.

FIG. 5. Ethanol production and D-xylose consumption during anaerobic D-xylose fermentation in the strain overexpressing the NADP-GAPDH (full symbols) and the corresponding control without NADP-GAPDH activity (open symbols). The concentrations are given in mM per g of dry weight.

FIG. 6. SDS PAGE of the purified NADP-GAPDH with histidine tag.

FIG. 7. Ethanol and xylitol production during anaerobic D-xylose fermentation in a strain with a ZWF1 deletion and overexpressing the NADP-GAPDH (triangles). The details are described in the example 5. For comparison the ethanol and xylitol production from FIG. 4 are included. The full symbols represent the ethanol production, the open symbols the xylitol production. The squares are for the control strain, the full circles for the strain overexpressing the NADP-GAPDH as described in the example 3.

FIG. 8. Ethanol production during anaerobic D-xylose fermentation with the strains described in the example 5.

FIG. 9. Xylitol production during anaerobic D-xylose fermentation in the strains as described in the example 5.

FIG. 10. D-xylose consumption during anaerobic D-xylose fermentation in the strains as described in the example 5.

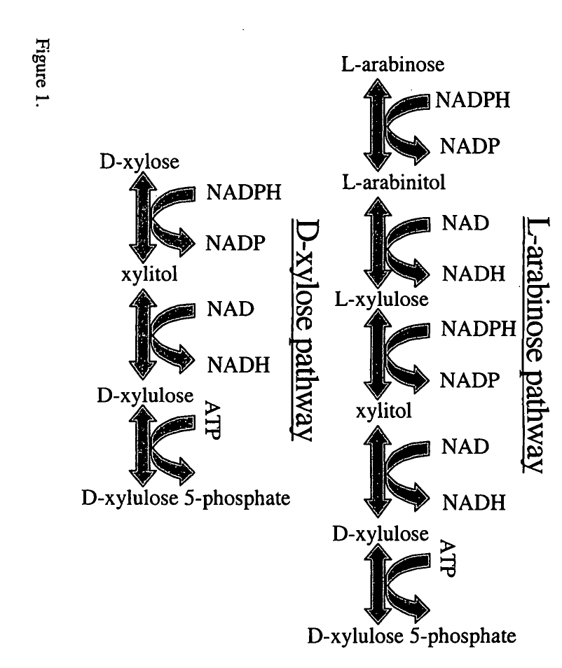

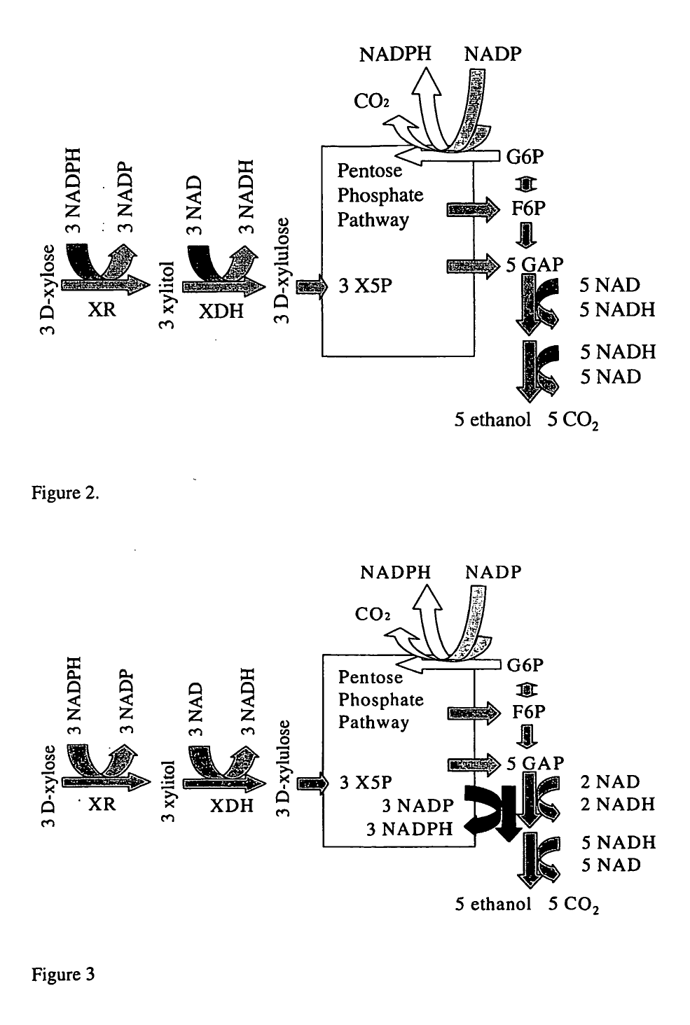

### DETAILED DESCRIPTION OF THE INVENTION

To look for possible proteins and their corresponding genes which could regenerate the redox cofactors NADP/NADPH in catabolic reactions such as pentose fermentation the following screening method for finding NADP/NADPH linked proteins and their corresponding genes can be used. In this screening method we used a Saccharomyces cerevisiae strain with a deletion in the gene coding for the phosphoglucose isomerase, PGI1. This deletion disables S. cerevisiae to grow on glucose (Boles et al., 1993). It is believed that this deletion leading to a lethal phenotype on glucose is related to an overproduction of NADPH in the oxidative part of the pentose phosphate pathway (Boles et al., 1993). Kluyveromyces lactis however can grow on glucose with a deletion in the phosphoglucose isomerase gene, i.e. it can cope with this NADPH overproduction (Gonzales Siso et al., 1996). We therefore transformed the S. cerevisiae strain with the deletion in phosphoglucose isomerase gene with a gene library from Kluyveromyces lactis and screened for growth on glucose. In this screening we found a DNA fragment that contained several open reading frames. A transposon was randomly inserted into the DNA fragment and those transposon insertions, which did not restore growth on glucose, were analysed. With this technique we identified the open reading frame which could restore growth on glucose. This open reading frame had high homology to NAD-GAPDH. We further investigated this open reading frame. For that purpose we overexpressed it and analysed the enzyme activity and found that it has activity with NADP. We further purified the enzyme after adding a histidine tag and found that the open reading frame codes for a protein which has a preference for NADP over NAD, i.e. it is not an NAD-GAPDH (EC 1.2.1.12) but an NADP-GAPDH (EC 1.2.1.13). This is surprising since there are no reports in the literature about NADP-GAPDH in eukaryotic organisms except plants, and there they are involved in photosynthesis, a reaction not carried out by yeasts. The NADP-GAPDH is encoded by the DNA sequence comprising SEQ ID No. 1.

Glyceraldehyde 3-phosphate dehydrogenases (GAPDH) are known as non-phosphorylating enzymes (GAPN, EC 1.2.1.8) and phosphorylating enzymes. For the phosphorylating enzymes nicotinamide dinucleotide (NAD) dependent enzymes (NAD-GAPDH, EC 1.2.1.12) and nicotinamide dinucleotide phosphate (NADP) dependent enzymes (NADP-GAPDH, EC 1.2.1.13) are known. The NAD-GAPDH is a glycolytic enzyme, which is highly conserved in prokaryotes and eukaryotes. NADP-GAPDH is known in bacteria (e.g. Koksharova et al. 1998, Fillinger et al. 2000). For plants an NADP-GAPDH, which is involved in the photosynthetic CO₂ assimilation and located in the chloroplasts, is known (Cerff 1982). The NADP-GAPDH of chloroplasts has the two subunits A and B (Shih et al. 1991, Baalmann et al. 1996). Other eukaryotic NADP-GAPDH are not known.

In the present invention we have two oxidoreductases with a common substrate but opposite coenzyme specificities, i.e. NAD-GAPDH (EC 1.2.1.12) and NADP-GAPDH (1.2.1.13). This however does not lead necessarily to cyclic oxidation-reduction reactions. As illustrated in the FIG. 3 for D-xylose fermentation, 3 moles of NADPH, which are used for the reduction of 3 moles of D-xylose, can be regenerated through the NADP-GAPDH. The NAD which is used by xylitol dehydrogenase and the NAD-GAPDH are regenerated by the alcohol dehydrogenase. The fermentation of 3 moles of D-xylose to 5 moles of ethanol and 5 moles of CO₂ is done without a cyclic transhydrogenase reaction, which makes the present invention different from the patent publication U.S. Pat. No. 5,830,716 and from the application WO 99/46363.

An NADP-GAPDH can be beneficial in processes where it is not desired to have the reduction of NADP to NADPH coupled to CO₂ production. One example is hexose fermentation. Because the microorganism grows during the fermentation it produces excesses of both NADH and NADP (Oura, 1972). Ethanol production is accompanied by glycerol production, which is required to reoxidise the excess NADH, and by the production of more than one mole of CO₂ per mole of ethanol, which is required to reduce the excess NADP. These reactions decrease the yield of ethanol on fermentable carbohydrate. With an NADP-GAPDH NADP can be reduced without extra to CO₂ production and by reducing NADP by using the glyceraldehyde 3-phosphate pool, less NADH is produced through the NAD-GAPDH and consequently less glycerol is produced, i.e. the introduction of NADP-GAPDH can increase the ethanol yield in hexose fermentation and decrease the formation of undesired sideproducts, glycerol and CO₂. The invention in this way makes the environmentally friendly production of fuel alcohol from hexose carbohydrates still more efficient and less polluting.

An NADP-GAPDH can also be beneficial in pentose fermentation. By the invention D-xylose and L-arabinose can be fermented to ethanol in a redox neutral way without creating a redox cofactor imbalance. In Examples 3 and 5 we show that D-xylose is fermented more efficiently to ethanol. Ethanol is produced from D-xylose with a higher yield and with less unwanted side products such as xylitol and CO₂.

This is shown in Example 3 where we show the effect of an NADP-GAPDH on anaerobic xylose fermentation. The strain overexpressing NADP-GAPDH produces, in molar ratios, about 30% less xylitol and about 40% less CO₂. As a consequence the ethanol is produced at a higher yield, i.e. from the same amount of D-xylose about 30% more ethanol is produced.

In addition to yield improvement and reduction of by-product formation, increased ability of the recombinant strain to carry out biotechnical processes may also be seen as improved rate of product formation, prolonged metabolic activity in process conditions or decreased demand for oxygen, all these factors increasing the efficiency of the process.

To further increase the ethanol yield, and decrease the CO₂ and xylitol yield, additional improvement strategies can be used. These include (1) decreasing the reactions competing for NADP with the NADP-linked GAPDH of our invention and (2) increasing the capacity or affinity of the NADP-GAPDH for NADP.

1) Decreasing Competing Reactions

NADPH regeneration through an NADP-GAPDH is not the only way to regenerate NADPH. Other pathways like through the oxidative part of the pentose phosphate pathway compete for the NADP. This NADPH regeneration is coupled to CO₂ production. It can be of further benefit to inhibit or delete this or similar pathways. We show in Example 5 that glucose 6-phosphate dehydrogenase competes for NADP and that the deletion of the corresponding gene, the ZWF1, together with the overexpression of the NADP-GAPDH has a further beneficial effect on ethanol production, i.e. ethanol is produced at a higher yield at the expense of unwanted side products such as xylitol or CO₂.

In Example 5 we demonstrate that decreasing the competing reactions for NADP we can further decrease the production of unwanted side products and thereby increase the ethanol yield. By deleting the gene for the glucose 6-phosphate. dehydrogenase, a reaction competing for NADP, and simultaneously overexpressing the NADP-GAPDH, we could decrease the production of unwanted xylitol by another 20%. Other reactions competing for NADP include the NADP dependent acetaldehyde dehydrogenase ALD6 and isocitrate dehydrogenases IDP1-3.

These and other reactions competing for NADP can be suppressed in a variety of ways. A gene encoding an enzyme catalysing the reaction can be deleted, as described in Example 5 for glucose 6-phosphate dehydrogenase. Such a gene can also be disrupted, so that it no longer produces a functional dehydrogenase. The promoter of the gene can also be altered (for example, by deletion of parts of the sequence upstream of the open reading frame) so that the expression level of the enzyme is decreased but not abolished. This can be advantageous if the reaction catalysed is beneficial to the microorganism so that e.g., complete suppression prevents growth of the microorganism. In practice, little experimentation is required, because if complete suppression prevents growth, this is immediately apparent and milder methods can be used with an obvious advantage. Similarly, mutations can be introduced to the active site of the competing enzyme, so that its catalytic efficiency is decreased, but not abolished. For example, a mutation that increased the Km for NADP of the competing enzyme would suffice, but it is not necessary to characterise the kinetic effects of such a mutation. The active site sequences of dehydrogenases are recognisable by those familiar with art

2) Increasing the Capacity of the NADP-GAPDH

To increase the capacity or affinity of the NADP-GAPDH the expression level can be increased or an NADP-GAPDH with a higher affinity towards NADPH can be used.

Decreasing competing reactions or increasing the capacity of the NADP-GAPDH are not only beneficial for pentose fermentation but also for the other examples where the NADP-GAPDH has a positive effect.

We describe here the introduction of an NADP-GAPDH from K. lactis to a strain of S. cerevisiae that contains the D-xylose pathway. For somebody knowledgeable in the art it is easy to find a similar enzyme from another fungi or other eukaryotic source. The introduction of an NADP-GAPDH can be beneficial independent of its source, whether it is bacterial, fungal or from another eukaryotic organism.

NADP-GAPDH are known from bacteria and from plants. In this invention we describe an NADP-GAPDH from fungi. An NADP-GAPDH can be generated e.g. through modification of the amino acid sequence of an NAD-GAPDH.

For example with the sequence of NADP-GAPDH disclosed herein comparison to the sequences of other dehydrogenases of known NAD and NADP specificity and some degree of amino acid identity, and in the best case to those for which the 3-D structure is known allows a person skilled in the art to predict the amino acids in the protein sequence which are responsible for the cofactor specificity. With this knowledge and using site directed mutagenesis the cofactor specificity can be changed, i.e. an NADP-GAPDH can be made by site directed mutagenesis from an NAD-GAPDH. It can be advantageous to create an NADP-GAPDH through mutagenesis in cases where the expression of a heterologous NADP-GAPDH is difficult. The desired change can also be done with random approaches.

One example how one can find in the sequence amino acids important for cofactor specificity of the enzyme is the following. Aligning the amino acid sequence of the NADP-GAPDH with those of glyceraldehyde 3-phosphate dehydrogenases from different organisms with different specificities and comparing this with the known structural information suggests that the amino acid 46 asparagine can be of importance (see also Fillinger et al., 2000). In all NAD-GAPDH the corresponding amino acid is the negatively charged aspartic acid. From the available structural information one would expect that the negatively charged phosphate of the NADP is in this area when NADP binds to the active site, i.e. NAD-GAPDH do not use NADP because of the unfavorable interaction between negative charges. By changing the negatively charged aspartic acid to a neutral residue, such as the asparagine disclosed here for the NADP-GAPDH of K. lactis, or to a positively charged amino acid one could change the specificity of an NAD-GAPDH so that it could also use NADP.

An NADP-GAPDH can also be beneficial in L-arabinose fermentation since the L-arabinose pathway creates a cofactor imbalance similar to the D-xylose pathway.

Polyhydroxyalkanoates (PHAs) are commercially produced to make biodegradable plastics, but prices are too high for widespread use except where this is enforced by legislation (e.g. in Germany). It is therefore desirable to improve the efficiency of the microbial processes producing PHAs. In the biosynthesis of PHAs, glucose is metabolised to acetyl-CoA, producing 2 NADH molecules/acetyl-CoA molecule, and the acetyl-CoA is then condensed to acetoacetyl-CoA which is reduced by NADPH to 3-hydroxybutyrylCoA. Synthesis of each molecule of 3-hydroxybutyrylCoA therefore produces 4 molecules of NADH and requires 1 molecule of NADPH. The 3-hydroxybutyrylCoA is then polymerised to polyhydroxybutyrate (PHB) or copolymerised with other acyl-CoAs such as propionyl-CoA to form mixed PHAs. The requirement for one NADPH molecule and production of 4 NADH molecules per monomer unit means that microorganisms synthesising PHAs need to divert part of their carbon flux through reactions such as glucose-6-phosphate dehydrogenase or isocitrate dehydrogenase in order to generate NADPH, with consequent excess production of CO₂ and waste of carbon source, as explained above. At the same time, NADH must be reoxidised, causing either further carbon losses or increased oxygen demand or both. By using a production microorganism transformed according to the present invention, so providing it with a novel mechanism that produces the NADPH at the expense of NADH (for reviews, see e.g. Anderson and Dawes [1990]; Poirier et al. [1995]). The waste of biomass as CO₂ is decreased, and so is the oxygen requirement, with consequent decreases the aeration costs.

The introduction of an NADP-GAPDH is not only beneficial in a strain of S. cerevisiae but also in other fungi, such as yeast species that naturally use pentoses. In any fungal species it is beneficial in D-xylose fermentation and in L-arabinose fermentation or in any biotechnological process where an imbalance of the redox cofactors imposes a hindrance. The fermentation products can be ethanol, lactate/lactic acid or other products.

It is well known to somebody skilled in the art that the amino acid sequence of an enzyme can be deliberately or accidentally (e.g. in PCR cloning) changed (e.g. parts deleted or added or amino acid changes introduced) so that the changed enzyme can still catalyse the same reaction as the original enzyme. The present invention can also be practised using recombinant DNA sequences that encode such ‘functionally active’ variants of NADP-GAPDH.

The present invention can also be practised by transforming a microorganism with a recombinant DNA molecule with a promoter different from the promoters used in the examples. It is not necessary that the transforming DNA molecule contains a nucleotide sequence encoding a complete functional enzyme. For example the beneficial effect can be obtained by transforming the natural host of an NADP-GAPDH with a DNA molecule that modifies the natural promoter, and so leads to an elevated expression level of the NADP-GAPDH.

Any method known in the art for transducing or transforming genes into the host is suitable for this invention and various types of vectors can be used, including autonomously replicating plasmid vectors or artificial chromosomes. Methods described in the art to integrate single or multiple copies of transforming genes into chromosomes in functional, expressible forms are also suitable for this invention.

It is envisaged in the invention that it can be advantageous in some cases to cause expression of the transformed genes only under specific culture conditions. For example it can be useful to first grow the organism to a certain cell density, and then cause expression of the transforming gene. Promoters are known that can be induced by changes in temperature or pH, by particular carbon or nitrogen sources or by the presence or absence in the medium of certain organic or inorganic substances such as phosphate or copper.

The present invention is further illustrated by the following examples, which are meant for illustration only and do not in any way limit the invention. If not otherwise indicated, all biotechnological procedures are carried out using methods conventional in the art.

### EXAMPLES

### Example 1

### Screening for Suitable Redox Enzymes

We used a screening system for NADP(H) related redox enzymes that is based on a deletion of the phosphoglucose isomerase gene in S. cerevisiae. A strain (Δpgil) with such a deletion is unable to grow on glucose, which is related to a lethal overproduction of NADPH (Boles et al., 1993). In Kluyveromyces lactis such a deletion does not lead to a similar phenotype (Gonzales Siso et al. 1996). We used a S. cerevisiae with a phosphoglucose isomerase deletion and screened a K. lactis genomic library for growth on glucose to find K. lactis genes that would allow the Δpgil mutant to grow on glucose. We found a gene for NADP linked GAPDH, as described above. Thus, this screening method provides genes suitable for practising the present invention.

Constructing the Host Strain for the Library Screening: Deleting the PGI1 Gene in S. cerevisiae:

The PGI1 gene of the S. cerevisiae haploid strain CEN.PK2 was deleted. A S. cerevisiae PGI1 fragment was obtained by PCR using the primers 3645 and 3646. The primer 3646 (5′-CGACCGGTCGACTACCAGCCTAAAAATGTC-3) had a SalI digestion site (underlined) to facilitate the cloning and the primer 3645 (5′-GGCACGCTGCAGAGAGCGATTTGTTCACAT-3′) had a PstI digestion site. The PGI1 fragment was digested with SalI and PstI and ligated into the pBluescript SK-vector (Stratagene). The resulting plasmid (B1186) was digested with EcoRI and BstBI to remove a 715 bp fragment from the middle of the PGI1 gene.

The HIS3 gene was obtained by DrdI digestion from the yeast expression vector pRS423. The HIS3 fragment was blunted with T4 DNA polymerase and ligated to the pBluescript SK-EcoRV site. This plasmid (B1185) was digested with EcoRI and ClaI and the 1.5 kb fragment carrying the HIS3 gene was ligated into EcoRI and BstBI digested B 1186 plasmid. The resulting plasmid was named B1187.

The PGI1+HIS3-fragment was released from the B1187 plasmid with SalI and MunI digestion and the S. cerevisiae strain CEN.PK2 was transformed with the fragment. The Li-acetate method (Hill et al., 1991; Gietz al., 1992) was used for the yeast transformation. The yeast transformants were confirmed by Southern blot-analysis using a fragment from the S. cerevisiae PGI1 gene as the probe. The resulting strain, CEN.PK2 Δpgil, was then used for the screening.

Construction and Screening of the K. lactis Genomic Library

The K. lactis genomic library was constructed into a yeast multicopy vector carrying the LEU2 marker gene as described by Brummer et al., 2001. The library was transformed into the CEN.PK2 Δpgil yeast strain. Transformants were plated on medium containing SC-leu+2% fructose+0.1% glucose. After 2 days cultivation 1.3*10⁶ transformants from the plates were pooled into 0.9% NaCl.

For the yeast library screening 6000 independent clones from the library were plated on medium containing SC-leu+2% fructose+0.1% glucose. After 3 days cultivation the colonies in the plates were replicated to SC-leu+0.1% glucose plates. The replica plates were cultivated for 9 days. 72 slowly growing colonies were streaked on SC-leu+0.1% glucose plates.

PCR-analysis was made to determine if the clones growing on glucose carried the K. lactis RAG2 gene coding for phosphoglucose isomerase. The PCR was made with specific primers 4719 and 4720 for the K. lactis RAG2. 5′-primer 4719 is 320 bp downstream from the ATG (5′-CACTGAAGGACGTGCTGTGT-3′) and 3′-primer 4720 is 1150 bp downstream from the ATG (5′-AGCTGGGAATCTGTGCAAGT-3′).

The PCR-analysis was made for 18 colonies. Six clones were found that did not carry the K. lactis RAG2 gene according to the PCR-analysis. Plasmid-DNA was extracted from these 6 clones and transformed into E. coli for further analysis.

The plasmids were retransformed to the CEN.PK2 Δpgil yeast strain and the transformants tested for growth on glucose. 2 clones were able to restore growth on glucose. Partial sequencing of the insert suggested that the two clones were identical. One of the plasmids was called B1513.

Identifying the Product of the Screening

The recovered plasmid had an insert of estimated 10 kb. A transposon was randomly inserted into the plasmid with the ‘Template generation system’ (Finnzymes). 10 different transposon insertions (as judged by PCR with primers from the transposon and the vector) were selected. They were then retransformed to the CEN.PK2 Δpgil strain tested for growth on 0.1% D-glucose. From strains, which were maintained on 2% D-fructose+0.05% D-glucose, but showed no growth on 0.1% D-glucose the plasmids were recovered and sequenced with primers of the transposon sequence. A plasmid that could not restore growth on D-glucose had a transposon inserted into an open reading frame with high homology to glyceraldehyde 3-phosphate dehydrogenase (GAPDH). The amino acid sequence of the enzyme which later turned out to be an NADP-GAPDH is presented by the SEQ ID No. 2. It is a protein with 356 amino acids having a molecular mass of 39030 Da. It is encoded by the open reading frame in the nucleotide sequence between nucleotides 384 and 1451 of the nucleotide sequence SEQ ID No. 1.

### Example 2

### Cloning and Expression of the GAPDH Homologue, Testing for NADPH-GAPDH Activity

Cloning the K. lactis GAPDH Homologue to the Yeast Expression Vector pYES2:

The GAPDH homologue was amplified by PCR from the plasmid B1513 from example 1 by using the following primers: GAPBAMH: AAGGATCCAAGCGTCTCCTTAAACACCAGC and GAPHIND: ATAAAGCTTAAGATGCCCGATATGACAAACGAATCTTC. The annealing temperature in the PCR was 65° C. The PCR product was digested with BamHI and HindIII and ligated to the corresponding sites in the multiple cloning site of the pYES2 vector (Invitrogen). The pYES2 is a yeast expression vector with a multiple cloning site between a galactose inducible promoter and terminator. The resulting vector was called B1612.

Expression of the K. lactis GAPDH Homologue in S. cerevisiae

The plasmid B1612 from above and as a control the plasmid pYES2 were transformed to the S. cerevisiae strain CEN.PK2. The resulting strains were grown on selective medium with 20 g/l D-glucose and 20 g/l D-galactose. Cells were harvested at an optical density of 1 and a cell extract prepared. The cell extract was prepared by vortexing 0.5 g cells (fresh weight) 500 mg glass beads (0.4 mm diameter) and 1 ml buffer (10 mM sodium phosphate pH 7.0 plus protease inhibitors). The extract was then used for an enzyme activity assay. The NADP-GAPDH enzyme activity was measured in a buffer containing 500 mM triethanol amine pH 7.8, 1 mM ATP, 2 MM MgCl₂, 0.2 mM NADPH, 3-phosphoglycerate kinase. To start the reaction, glycerate 3-phosphate was added at a final concentration of 5 mM. The activity was calculated from the decrease in NADPH absorbance at 340 nm. We found an NADPH-GAPDH activity of 0.05 nkat per mg of extracted protein. In the control, where the empty pYES2 plasmid was transformed we found 0.006 nkat per mg.

### Example 3

### Effect of K. lactis GAPDH Homologue on D-Xylose Fermentation in an S. cerevisiae Strain

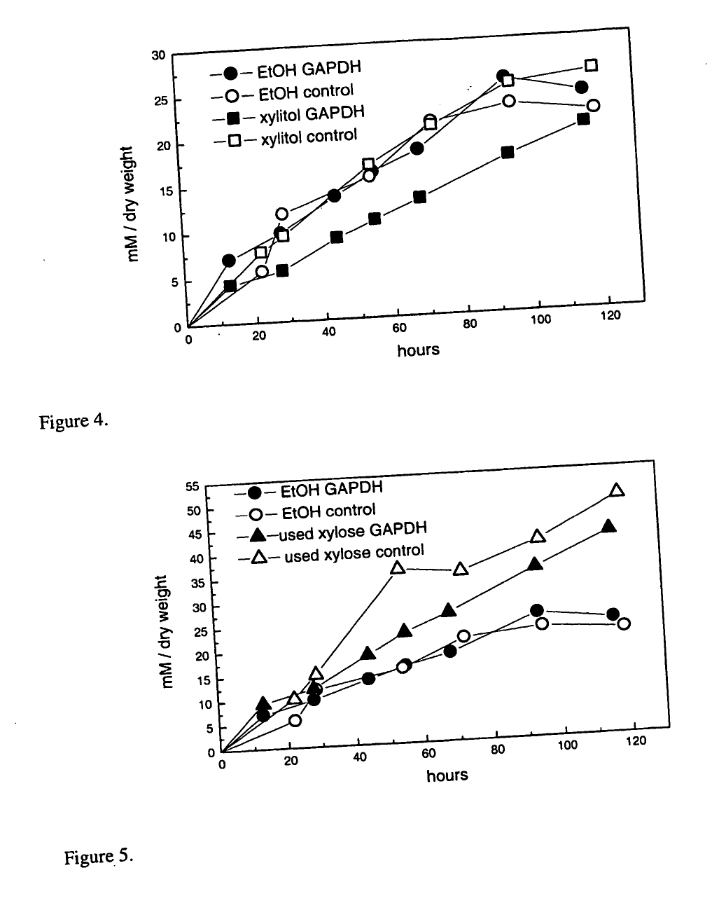

For the D-xylose fermentation the NADP-GAPDH gene was ligated to a yeast expression vector with ADH1 promoter. Therefore the NADP-GAPDH was amplified by PCR as described in the example 2 except that the following primers, each of them containing a BamHI restriction site, were used: (BamHI sites are underlined) AAGGATCCAAGATGCCCGATATGACAAACGAATCTTC and AAGGATCCAAGCGTCTCCTTAAACACCAGC. The PCR product was then cloned to a TOPO vector (Invitrogen) and the 1 kb BamHI fragment from the resulting vector ligated to the BamHI site of the pVT102U (Vernet et al 1987). The resulting vector (B1731) was then transformed to a S. cerevisiae strain (H2217, Aristidou et al 1999), which overexpressed the enzymes of the xylose pathway, i.e. xylose reductase (XR), xylitol dehydrogenase (XDH) and xylulokinase (XK) were integrated into the genome. As a control we used the same strain, except that it lacked the NADP-GAPDH. Instead it contained the empty vector pVT102U. With both strains pure D-xylose was fermented under anaerobic conditions. The cells were first grown in a medium with yeast nitrogen base (Difco) and all amino acids except uracil and 30 g/l D-glucose as a carbon source in a volume of 1.6 l at 30° C., pH 5.0 and an airflow rate of 2 l/min. After 48 hours the biomass was 3 to 4 g/l and the ethanol concentration between 0.5 and 1 g/l when 0.4 l of a D-xylose solution was added so that the final concentration of D-xylose was 50 g/l. The gas flow was changed to nitrogen at a flow rate of 0.1 l/min. Liquid samples were taken and analysed for dry weight and by HPLC for ethanol, xylose and xylitol and other components. The outlet gas was analysed by mass spectroscopy. The results are in the FIGS. 3 and 4. The main products of such a fermentation are xylitol, ethanol and CO₂. When introducing the NADP-GAPDH the molar ratio of produced ethanol to xylitol was increased. Without the NADP-GAPDH the molar concentrations of xylitol and ethanol are similar. With the introduction of the NADP-GAPDH the production of xylitol is decreased by about 30% (FIG. 4). The ethanol yield on D-xylose is also affected. The maximal theoretical yield is 1.67 mol ethanol per mol D-xylose. In the control we find a molar ratio of ethanol produce to D-xylose consumed of 0.44 which is 26% of the theoretical yield, with the NADP-GAPDH the molar ratio of D-xylose consumed to ethanol produced is 0.57 which is 34% of the theoretical yield (FIG. 4). Also the CO₂ yield is affected. We analysed the CO₂ production in the period from 30 to about 90 hours and compared it with the ethanol production in the same period. With the NADP-GAPDH the molar ratio of CO₂ over ethanol is 1.15 as compared to 1.91 in the control.

### Example 4

### The Purified NADP-GAPDH

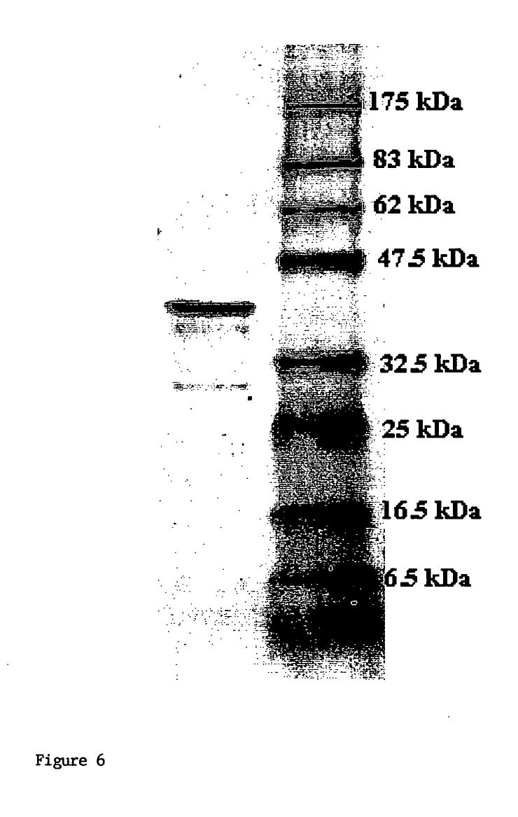

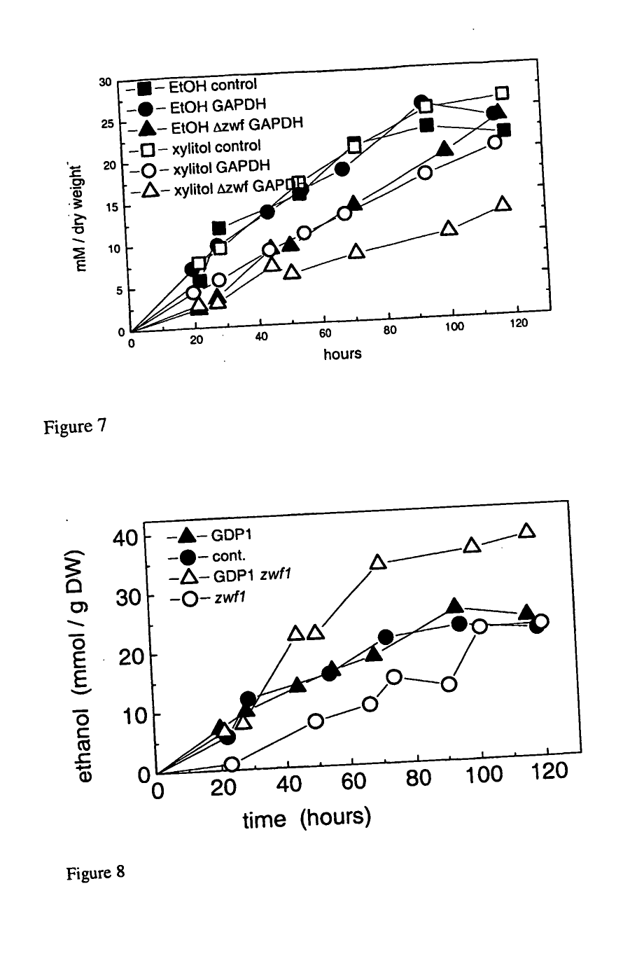

To purify the NADPH-GAPDH we overexpressed it in yeast with an additional tag of 6 histidines at the N-terminus of the protein. It was cloned by PCR similar to the Example 2 and 3. The primers were AAGGATCCAAGATGCCCGATATGACAAACGAATCTTC for the start of the gene with a BamHI restriction site and AAGGATCCTTAATGATGATGATGATGATGAACACCAGCTTCGAAGTCCT TTTGAGCC for the end of the gene with the introduction of 6 histidines and a BamHI restriction site underlined. The PCR product was first cloned to a TOPO vector (Invitrogen) and the BamHI fragment from the TOPO vector then ligated to the BglII site of a yeast expression vector with a PG1 promoter (B1181). This yeast expression vector was made by digesting the yeast expression vector pMA91 (Mellor et al., 1983) with HindIII and ligating the resulting 1.8 kb fragment, containing the PGK1 promoter/terminator with a BglII cloning site, to the HindIII site of the YEplac195 vector (Gietz and Sugino, 1988). The plasmid was then transformed to a yeast strain with a mutation in the phosphoglucose isomerase gene. The plasmid could restore growth on glucose showing that the histidine tag did not affect the enzyme activity. The His-tagged protein was then purified with a NiNTA column (Qiagen). The so purified protein was then applied to a SDS-PAGE as shown in FIG. 6. The enzyme is almost pure. An estimated 80 to 90% of the protein in the SDS-PAGE is in a single band of about 40 kDa. The activity was measured as described in the example 2 with 200 μM NADPH or 200 μM NADH. With NADPH we found an activity of 140 nkat/mg, with NADH an activity of 47 nkat/mg.

### Example 5

### Effect of Deletion of the Glucose 6-Phosphate Dehydrogenase in the Presence of NADP-GAPDH on D-Xylose Fermentation

Construction of the Δzwf1 Deletion Strain

The ZWF1 gene coding for the glucose 6-phosphate dehydrogenase (G6PDH) was obtained by PCR using S. cerevisiae genomic DNA as a template. Specific primers 3994 (5′-GCTATCGGATCCAAGCTTAGGCAAGATGAGTGAAGGTT-3′) and 4006 (5′-GCTATCGGATCCAAGCTTAGTGACTTAGCCGATAAATG-3′) were used. Both the primers had BamHI and HindIII sites to facilitate the cloning. The restriction sites are underlined. The ZWF1 fragment obtained from the PCR was digested with BamHI and ligated into the pBluescript SK-plasmid (Stratagene). The resulting plasmid B1768 was digested with BglII. In the digestion a 1063 bp fragment was released from the middle of the ZWF1 gene. The digested vector was blunted with Mung Bean Nuclease. The HIS3 marker gene was obtained from the pRS423 plasmid (Christianson et al., 1992) by BsmBI and DraIII digestion. The 1591 bp fragment containing the HIS3gene was blunted with Mung Bean Nuclease and ligated into the BglII digested and blunted B 1768 vector. The resulting plasmid was named B1769. The ZWF1 deletion cassette was released from the B1769 plasmid with BamHI digestion and the S. cerevisiae strain H2217 (see example 3) was transformed with the fragment by Li-acetate method. The deletion of the ZWF1 gene was confirmed by PCR-analysis, by Southern blot-analysis and by G6PDH enzyme activity assay.

The cell extracts for the G6PDH enzyme activity measurement were prepared by disrupting the yeast cells in 10 mM Na-phosphate pH 7.0 buffer using glass beads. The protease inhibitors PMSF (final concentration 1 mM) and pepstatin A (0.01 mg/ml) were added into the extraction buffer. The activity was measured with Cobas Mira analyser (Roche). The activity was measured in buffer containing 10 mM Na-phosphate pH 7.0 and 1 mM NADP and 10 mM G6PDH was used as start reagent. No G6PDH activity was found in the Δwf1 deletion strain.

The ZWF1 gene coding for the glucose 6-phosphate dehydrogenase was deleted in a S. cerevisiae strain in which the genes for xylose reductase, xyhtol dehydrogenase and xylulokinase were integrated into the genome as described in the example 3.

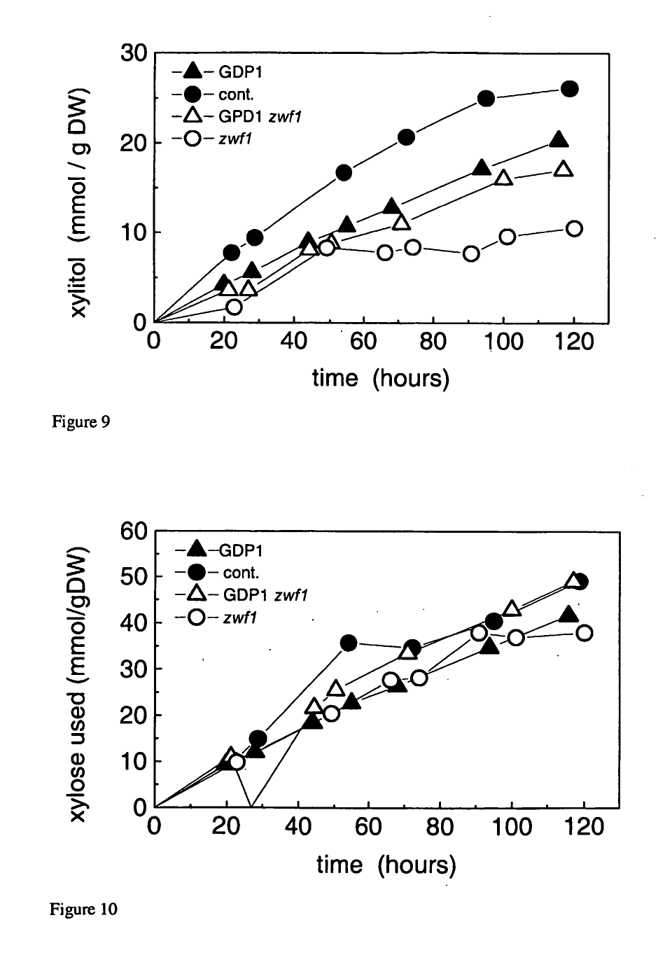

The resulting strain was then transformed with a multicopy expression vector with the NADP-GAPDH under the PGK1 promoter. To make this expression vector the 1 kb BamHI fragment with the NADP-GAPDH as described in the example 3 was ligated to the BglII site of the B1181 vector as described in the example 4. A control strain was made with the empty vector B1181 in the zwf1 deletion strain. Four strains were compared strain 1: GDP1, the strain expressing the gene for the GAPDH; strain 2: control, the strain with the empty vector; strain 3: GDP1 Δzwf1, The strain expressing the the gene for the GAPDH in the background of a zwf1 deletion and strain 4: Δzwf1, the strain with the zwf1 deletion and an empty plasmid. All strains have also the genes coding for D-xylose reductase, xylitol dehydrogenase and xylulokinase integrated into the genome. These strain were then used to ferment D-xylose under anaerobic conditions as described in the example 3. The result is summariesed in the table 1 and 2 and FIGS. 8, 9 and 10.

TABLE 1
Summary of the fermentation described in Example 5. The D-xylose
consumption during the 120 hour period of anaerobis fermentation is
compared with the production of ethanol, xylitol and CO2 during
the same period.

| | mM xylose | mM | mM | mM | |
|---|---|---|---|---|---|
| | used | ethanol | xylitol | CO₂ | recovery |
| Strain | (c-mol %) | (c-mol %) | (c-mol %) | (c-mol %) | c-mol % |
| control | −200 (−100%) | 90 (18%) | 110 (53%) | 225 (22%) | 95% |
| GDP1 | −135 (−100%) | 76 (23%) | 65 (48%) | 150 (22%) | 95% |
| Δzwf1 | −175 (−100%) | 100 (24%) | 50 (28%) | 160 (18%) | 70% |
| GDP1 | −98 (−100%) | 100 (41%) | 34 (34%) | 125 (25%) | 100% |
| Δzwf1 | | | | | |

TABLE 2
The molar ratios of ethanol/xylitol, ethanol/D-xylose used and CO₂/
ethanol after the 120 h period of fermentation of example 5

| | ethanol/xylitol | ethanol/xylose | |
|---|---|---|---|
| strain | mol/mol | used | CO₂/ethanol mol/mol |
| GDP1 | 1.2 ± 0.15 | 0.56 ± 0.05 | 2 ± 0.2 |
| control | 0.9 ± 0.1 | 0.45 ± 0.04 | 2.5 ± 0.2 |
| GDP1 Δzwf1 | 3 ± 0.3 | 1.02 ± 0.1 | 1.3 ± 0.1 |
| Δzwf1 | 2 ± 0.2 | 0.57 ± 0.06 | 1.6 ± 0.1 |

### Example 6

Anaerobic shake flask cultivation on D-glucose D-xylose mixtures with ¹⁴C-labelled D-xylose. The initial biomass was 0.365 g/l dry weight. 100 ml of yeast suspension in a D-glucose, D-xylose mixture as indicated, was stirred with a magnetic stirrer in a 100 ml erlenmeyer with a waterlock to ensure anaerobiosis at 30° C. Fermentation was 75 hours. After the fermentation the ethanol was distilled from 50 ml of medium and filled up to a volume of 50 ml. The total ethanol was then measured by measuring the density of the distillate with the Anton-Paar DMA58 density meter. The ethanol derived from D-xylose was estimated from the radioactivity of the distillate.

All strains are derived from a strain with XR, XDH and XK integrated and are described in the previous example

TABLE 3
Summarised results of Example 6. The dry weight, total ethanol and ethanol from
D-xylose after the fermentation period is given for the various strains and initial
sugar compositions. The ‘% of theoretical from xylose’ is the fraction of ethanol
derived from xylose given in % compared to the theoretical yield which is 5/3 mol
of ethanol per mol of xylose if all D-xylose was consumed.

| | | | | | % of | |
|---|---|---|---|---|---|---|
| | | | | ethanol | theoretical | |
| | | dry | total | from D- | from xylose | ethanol from |
| sugar | | weight | ethanol | xylose | * | xylose % |
| composition | strain | g/l | mM | mM | ** | change |
| 2.4% D-glucose | GDP1, | 2.08 | 310 | 22.8 | 34 | +42% |
| 0.6% D-xylose | Δzwf1 | | | | | |
| | Δzwf1 | 1.94 | 270 | 12.3 | 19 | −21% |
| | control | 2.17 | 278 | 16 | 24 | 0 |
| | GDP1 | 2.14 | 279 | 17.2 | 26 | +8% |
| 2.4% D-glucose | GDP1, | 2.19 | 322 | 37 | 28 | +39% |
| 1.2% D-xylose | Δzwf1 | | | | | |
| | Δzwf1 | 2.00 | 275 | 20.4 | 15.5 | −23% |
| | control | 2.21 | 286 | 26.5 | 20.1 | 0 |
| | GDP1 | 2.34 | 288 | 29.5 | 22.4 | +11% |
| 2.4% D-glucose | GDP1, | 2.22 | 324 | 44.1 | 16.8 | +45% |
| 2.4% D-xylose | Δzwf1 | | | | | |
| | Δzwf1 | 2.08 | 275 | 25.35 | 9.6 | −17% |
| | control | 2.09 | 285 | 30.65 | 11.6 | 0 |
| | GDP1 | 2.27 | 293 | 41.5 | 15.8 | +36% |

* theoretical from glucose is 264 mM ethanol
** theoretical from xylose is 66 mM, 132 mM and 263 mM respectively

References
Aristidou, A., Londesborough, J., Penttilä, M., Richard, P., Ruohonen, L., Söderlund, H., Teleman, A. and Toivari, M., Transformed microorganisms with improved properties PCT/F199/00185. WO 99/46363 (1999)
Anderson A J and Dawes E A (1990). Occurrence, metabolism, metabolic role, and industrial uses of bacterial polyhydroxyalkanoates. Microbiol. Reviews 54: 450-472.
Baalmann, E., Scheibe, R., Cerff, R. and Martin, W. Functional studies of chloroplast glyceraldehyde-3-phosphate dehydrogenase subunits A and B expressed in Escherichia coli: formation of highly active A4 and B4 homotetramers and evidence that aggregation of the B4 complex is mediated by the B subunit carboxy terminus. Plant Mol. Biol. 32 (3), 505-513 (1996)
Boles, E., Lehnert, W. and Zimmerman, F. K. The role of NAD-dependent glutamate dehydrogenase in restoring growth on glucose of a Saccharomyces cerevisiae phosphoglucose isomerase mutant. Eur. J. Biochem. (1993) 712, 469-477
Brummer, M. H., Richard, P., Sundqvist, L., Väänänen, R. and Keränen, S. The GDI1 genes from Kluyveromyces lactis and Pichia pastoris: cloning and functional expression in Saccharomyces cerevisiae. Yeast 2001, 10:897-902
Cerff, R. (1982) in: Methods in Chloroplast Molecular Biology (Edelman, M., Hallick, R. B. and Chua, N.-H., eds) pp683-694, Elsevier/North Holland, Amsterdam
Christianson, T. W., R. S. Sikorski, M. Dante, J. H. Shero, and P. Hieter. 1992. Multifunctional yeast high-copy-number shuttle vectors. Gene. 110:119-122
Fillinger S, Boschi-Muller S, Azza S, Dervyn E, Branlant G, Aymerich S., Two glyceraldehyde-3-phosphate dehydrogenases with opposite physiological roles in a nonphotosynthetic bacterium. J Biol Chem 2000 May 12; 275(19): 14031-14037
Gietz, D., St_Jean, A., Woods, R. A. and Schiestl, R. H. (1992) Improved method for high efficiency transformation of intact yeast cells. Nucleic Acids Research, 20, 1425.
Gietz R D and Sugino A (1988). New yeast-Escherichia coli shuttle vectors constructed with in vitro mutagenized yeast genes lacking six-base pair restriction sites. Gene 74: 527-534.
Goffrini, P., Wesolowski-Louvel, M. and Ferrero, I. (1991) A phosphoglucose isomerase gene is involved in the Rag phenotype of the yeast Klyveromyces lactis. Mol. Gen. Genet. 228, 401-409
Gonzales Siso, M. I., Freire Picos, M. A. and Cerdan M. E. (1996) Reoxidation of the NADPH produced by the pentose phosphate pathway is necessary for the utilisation of glucose by Kluyveromyces lactis rag2 mutants. FEBS Letters 387, 7-10
Hill, J., Donald, K. A., Griffiths, D. E. and Donald, G. (1991) DMSO-enhanced whole cell yeast transformation. Nucleic Acids Research, 19, 5791.
Jeffries, T. W. and Jin Y.-S. (2000) Ethanol and thermotolerance in the bioconversion of xylose by yeasts in: Advances in applied microbiology, Volume 47, Academic press
Koksharova O, Schubert M, Shestakov S, Cerff R., Genetic and biochemical evidence for distinct key functions of two highly divergent GAPDH genes in catabolic and anabolic carbon flow of the cyanobacterium Synechocystis sp. PCC 6803. Plant Mol Biol 1998 January;36(1):183-194
Mellor J, Dobson M J, Roberts, N A, Tuite M F, Emtage J S, White S, Lowe P A, Patel T, Kingsman A J and Kingsman S M (1983). Efficient synthesis of enzymatically active calf chymosin in Saccharomyces cerevisiae. Gene 24: 1-14.
Oura, E. (1972) The effect on aeration on the growth energetics and biochemical composition of baker's yeast. Doctoral Dissertation, University of Helsinki, p.30
Poirier Y, Nawrath C and Somerville C (1995). Production of polyhydroxyalkanoates, a family of biodegradable plastics and elastomers, in bacteria and plants. BIO/Technology 13: 142-150.
Shih, M. C., Heinrich, P. and Goodman, H. M. Cloning and chromosomal mapping of nuclear genes encoding chloroplast and cytosolic glyceraldehyde-3-phosphate-dehydrogenase from Arabidopsis thaliana [published erratum appears in Gene 1992 Oct. 1; 119(2):317-9] Gene 104 (2), 133-138 (1991)
Toivari, M. H., Aristidou, A., Ruohonen, L. & Penttilä, M. (2001). Conversion of xylose to ethanol by recombinant Saccharomyces cerevisiae: Importance of xylulokinase (XKS1) and oxygen availability. Metab. Eng. 3, 236-249.
Vernet, T., Dignard, D. and Thomas, D. Y. (1987) A family of yeast expression vectors containing the phage f1 intergenic region. Gene, 52, 225-233

1. A fungal microorganism having an increased ability to carry out biotechnological processes, said microorganism having been transformed with a DNA sequence encoding an NADP-linked glyceraldehyde 3-phosphate dehydrogenase.

2. The fungal microorganism according to claim 1, wherein said NADP-linked glyceraldehyde 3-phosphate dehydrogenase is of fungal origin.

3. The fungal microorganism of claim 2, wherein the DNA sequence encoding the NADP-linked glyceraldehydes 3-phosphate dehydrogenase comprises SEQ ID No. 1 or a functional variant thereof.

4. The fungal microorganism according to claim 1, wherein the biotechnological process includes L-arabinose or D-xylose as carbon sources.

5. The fungal microorganism according to claim 1, wherein the microorganism is further manipulated to suppress other NADP/NADPH linked reactions.

6. A method of producing useful industrial product(s) from carbon sources, comprising producing from the carbon sources by a fungal microorganism according to claim 1.

7. A method of producing useful industrial product(s) from carbon sources according to claim 6, wherein the carbon sources contain pentose.

8. A method of claim 6 wherein the useful industrial product is ethanol.

9. A method of producing polyhydroxybutyrate from carbon sources containing glucose, comprising the use of a fungal microorganism according to claim 1.

10. The fungal microorganism of claim 1, wherein the DNA sequence encoding the NADP-linked glyceraldehydes 3-phosphate dehydrogenase comprises SEQ ID No. 1 or a functional variant thereof.

11. The fungal microorganism according to claim 2, wherein the biotechnological process includes L-arabinose or D-xylose as carbon sources.

12. The fungal microorganism according to claim 3, wherein the biotechnological process includes L-arabinose or D-xylose as carbon sources

13. The fungal microorganism according to claim 2, wherein the microorganism is further manipulated to suppress other NADP/NADPH linked reactions.

14. The fungal microorganism according to claim 3, wherein the microorganism is further manipulated to suppress other NADP/NADPH linked reactions.

15. The fungal microorganism according to claim 4, wherein the microorganism is further manipulated to suppress other NADP/NADPH linked reactions.

16. A method of producing useful industrial product(s) from carbon sources, comprising producing from the carbon sources by a fungal microorganism according to claim 2.

17. A method of producing useful industrial product(s) from carbon sources, comprising producing from the carbon sources by a fungal microorganism according to claim 3.

18. A method of producing useful industrial product(s) from carbon sources, comprising producing from the carbon sources by a fungal microorganism according to claim 4.

19. A method of producing useful industrial product(s) from carbon sources, comprising producing from the carbon sources by a fungal microorganism according to claim 5.

20. A method of claim 7 wherein the useful industrial product is ethanol.

21. A method of producing polyhydroxybutyrate from carbon sources containing glucose, comprising the use of a fungal microorganism according to claim 2.

22. A method of producing polyhydroxybutyrate from carbon sources containing glucose, comprising the use of a fungal microorganism according to claim 3.

23. A method of producing polyhydroxybutyrate from carbon sources containing glucose, comprising the use of a fungal microorganism according to claim 4.

24. A method of producing polyhydroxybutyrate from carbon sources containing glucose, comprising the use of a fungal microorganism according to claim 5.


SEQUENCE LISTING (13 sequences)

SEQ ID NO: 1 - DNA, 1780 bp, Kluyveromyces lactis strain CBS2359
ggatgtacatgccatgggcaatttaagacgaaatcatctaaccggtaaacgactggtgtttttttttttggttgatggtttccttaaattatacaaatccagagataatcatatctaattaaccgctgtctgctctgctttcactgctttcgcatcaccgttttcttctttttaccttttttaagaaataaagatcttactggtactgctacgctaggtcatccacttgtgatttaataacattgacattagaaaacttctttattttctaatatttactggcacatattaccgctattattattatttgagagaaaagttactttttttctttaaaccggaccttttaaaatctcacagaattgatactgtcacttttttagatgcccgatatgacaaacgaatcttcttctaagccagctcaaattaacattggtatcaatggttttggtagaatcggtagattggttctacgtgctgctttgacgcacccagaagttaaggtcagattaatcaataatccatccacaacaccagaatacgctgcttatttgttcaaatacgattctactcacggcaagtatcgtggtgaagttgaattcgacgatgaacgtatcatcattcaaaatgaccatgtttcggctcatatccctctatctcattttagggaaccagagcgtatcccatgggcttcctacaacgtcgattatgtaattgactcaaccggtgtcttcaaggaagtcgatacagcctctagacataaaggtgtcaaaaaagttatcattactgctccatcaaagaccgcgccaatgtacgtctatggtgttaaccacgttaaatacaacccattgacggatcacgtggtctctaatgcctcctgtactaccaactgtttggctccgttggttaaggctttggacgatgagttcggtatcgaagaagccttgatgacaactattcatgcaactactgcttctcaaaagactgtcgatggtaccagttctggtggtaaggactggagaggcggtagatcttgccagggaaatatcattccttcatctactggtgcagctaaggctgtagggaaaatcttgcctgaacttaatggtaagatcaccggtatgtctataagagtcccaacaattaatatttccctggttgacttgacattccgtacagcaaagaaaacttcttacgatgacattatgaaggccctagaacaaagatctcgcagcgatatgaagggtgttttgggtgttaccaaagacgccgttgtgtcctctgacttcacatccgattcacgttcatctattgttgatgccaaggccggtattgaattgaacgaccattttttcaaggtcctttcttggtatgataatgaatatggttactcttcaagagtggttgatttatccattttcatggctcaaaaggacttcgaagctggtgtttaaggagacgctttttttgtttgatttcaaaatcccagtagactgacaaaaaagtactcttcgtttgatgaagactattctcaaaagttttcgacaatttacaaaattatgatggtacttcaaaacaataccttttgtacctccatccgccattattatttaatattcacagcaatttaaagatattctatttactaaaggatatcttcagaaaatttcttaacacatatagacaagttaaataaaaacccataccatgtaagcataatgaagtatacgatcttaatattaattggtaataatgtatttatttaacctcgaaggcactt

SEQ ID NO: 2 - protein, 356 aa, Kluyveromyces lactis strain CBS2359
MPDMTNESSSKPAQINIGINGFGRIGRLVLRAALTHPEVKVRLINNPSTTPEYAAYLFKYDSTHGKYRGEVEFDDERIIIQNDHVSAHIPLSHFREPERIPWASYNVDYVIDSTGVFKEVDTASRHKGVKKVIITAPSKTAPMYVYGVNHVKYNPLTDHVVSNASCTTNCLAPLVKALDDEFGIEEALMTTIHATTASQKTVDGTSSGGKDWRGGRSCQGNIIPSSTGAAKAVGKILPELNGKITGMSIRVPTINISLVDLTFRTAKKTSYDDIMKALEQRSRSDMKGVLGVTKDAVVSSDFTSDSRSSIVDAKAGIELNDHFFKVLSWYDNEYGYSSRVVDLSIFMAQKDFEAGV

SEQ ID NO: 3 - DNA, 30 bp, Artificial Sequence
  Primer 3646
cgaccggtcgactaccagcctaaaaatgtc

SEQ ID NO: 4 - DNA, 30 bp, Artificial Sequence
  Primer 3645
ggcacgctgcagagagcgatttgttcacat

SEQ ID NO: 5 - DNA, 20 bp, Artificial Sequence
  Primer 4719
cactgaaggacgtgctgtgt

SEQ ID NO: 6 - DNA, 20 bp, Artificial Sequence
  Primer 4720
agctgggaatctgtgcaagt

SEQ ID NO: 7 - DNA, 30 bp, Artificial Sequence
  Primer GAPBAMH
aaggatccaagcgtctccttaaacaccagc

SEQ ID NO: 8 - DNA, 38 bp, Artificial Sequence
  Primer GAPHIND
ataaagcttaagatgcccgatatgacaaacgaatcttc

SEQ ID NO: 9 - DNA, 37 bp, Artificial Sequence
  Primer used for amplification of NADP-GAPDH
aaggatccaagatgcccgatatgacaaacgaatcttc

SEQ ID NO: 10 - DNA, 30 bp, Artificial Sequence; identical to SEQ ID NO: 7
  Primer used for amplification of NADP-GAPDH

SEQ ID NO: 11 - DNA, 57 bp, Artificial Sequence
  Primer used for amplification of NADP-GAPDH
aaggatccttaatgatgatgatgatgatgaacaccagcttcgaagtccttttgagcc

SEQ ID NO: 12 - DNA, 38 bp, Artificial Sequence
  Primer 3994
gctatcggatccaagcttaggcaagatgagtgaaggtt

SEQ ID NO: 13 - DNA, 38 bp, Artificial Sequence
  Primer 4006
gctatcggatccaagcttagtgacttagccgataaatg